(19) United States
(12) Patent Application Publication
(10) Pub. No.: US 2026/0042722 A1
(43) Pub. Date: Feb. 12, 2026

(54) METHOD FOR PRODUCING ISOMERS OF 1,2-CHLORO-2,3,3-TRIFLUORO-1-PROPENE (HCFO-1233yd)

(71) Applicant: Honeywell International Inc., Charlotte, NC (US)

(72) Inventors: Haiyou Wang, Amherst, NY (US); Xinrui Yu, Furlong, PA (US)

(21) Appl. No.: 19/283,670

(22) Filed: Jul. 29, 2025

Related U.S. Application Data
(60) Provisional application No. 63/680,512, filed on Aug. 7, 2024.

(51) Int. Cl. C07C 17/358; C07C 17/38

(52) U.S. Cl. CPC C07C 17/358; C07C 17/38; C07B 2200/09; C07C 2523/26; C07C 2527/126; C07C 2527/132

(57) ABSTRACT

Production of HCFO-1233yd blends with broad range of Z-isomer and/or E-isomer is achieved through isomerization reactions under suitable reaction conditions. Methods of producing cis-HCFO-1233yd(Z) may include providing a reactant stream containing at least 50% HCFO-1233yd(E), vaporizing at least a portion of HCFO-1233yd(E) in the reactant stream, converting at least a portion of HCFO-1233yd(E) to HCFO-1233yd(Z) in a reactor, and separating and recovering a composition comprising HCFO-1233yd(Z) or a mixture of HCFO-1233yd(E) and HCFO-1233yd(Z). The reaction may be conducted in the presence of a catalyst.

### CROSS-REFERENCE TO RELATED APPLICATIONS

The present application claims the benefit under 35 U.S.C. § 119(e) of U.S. Provisional Patent Application Ser. No. 63/680,512, filed on Aug. 7, 2024, the entire disclosure of which is incorporated by reference herein.

### FIELD

The present disclosure is directed to a method for producing 1-chloro-2,3,3-trifluoro-1-propene (HCFO-1233yd), including trans-1-chloro-2,3,3-trifluoro-1-propene (HCFO-1233yd(E)) and/or cis-1-chloro-2,3,3-trifluoro-1-propene (HCFO-1233yd(Z)).

### BACKGROUND

Fluorocarbon fluids have properties that are desirable for use as heat transfer media, immersion coolants, liquid or gaseous dielectrics, industrial refrigerants, and other applications.

1-chloro-2,3,3-trifluoro-1-propene (HCFO-1233yd) has recently found increased utility for a variety of uses. HCFO-1233yd may exist as a mixture of two geometric isomers, the E- or trans isomer and the Z- or cis isomer, which may be used separately or together in various proportions. Both isomers can be used to make various blends as solvents. Potential end use applications of HCFO-1233 include degreasing, de-fluxing of printed wiring assemblies, and carrier solvent for silicone oils, etc.

Improved methods to produce HCFO-1233yd and for converting HCFO-1233yd(E) to HCFO-1233yd(Z) and/or from HCFO-1233yd(Z) to HCFO-1233yd(E) are desired.

### SUMMARY

The present disclosure provides compositions including HCFO-1233yd and methods for converting HCFO-1233yd(E) to HCFO-1233yd(Z) and/or from HCFO-1233yd(Z) to HCFO-1233yd(E) in an efficient and cost-effective manner.

In one form thereof, the present disclosure provides a method for producing HCFO-1233yd(Z), comprising providing a reactant stream containing at least 50% HCFO-1233yd(E), vaporizing at least a portion of HCFO-1233yd(E) in the reactant stream, converting at least a portion of HCFO-1233yd(E) to HCFO-1233yd(Z) in a reactor, and separating and recovering a composition comprising HCFO-1233yd(Z) or a mixture of HCFO-1233yd(E) and HCFO-1233yd(Z).

In another form thereof, the present disclosure provides a method for producing HCFO-1233yd(E), comprising providing a reactant stream containing at least 50% HCFO-1233yd(Z), vaporizing at least a portion of HCFO-1233yd(Z) in the reactant stream, converting at least a portion of HCFO-1233yd(Z) to HCFO-1233yd(E) in a reactor, and separating and recovering a composition comprising HCFO-1233yd(E) or a mixture of HCFO-1233yd(E) and HCFO-1233yd(Z).

### BRIEF DESCRIPTION OF THE DRAWINGS

FIG. 1 is a process flow diagram of a process converting trans-1-chloro-2,3,3-trifluoro-1-propene (HCFO-1233yd(E)) to cis-1-chloro-2,3,3-trifluoro-1-propene (HCFO-1233yd(Z)) (“Process 1”).

FIG. 2 is a process flow diagram of a process for converting trans-1-chloro-2,3,3-trifluoro-1-propene (HCFO-1233yd(E)) to cis-1-chloro-2,3,3-trifluoro-1-propene (HCFO-1233yd(Z)) (“Process 1”).

FIG. 3 is a process flow diagram of a process for converting cis-1-chloro-2,3,3-trifluoro-1-propene (HCFO-1233yd(Z)) to trans-1-chloro-2,3,3-trifluoro-1-propene (HCFO-1233yd(E)) (“Process 2”).

FIG. 4 is a process flow diagram of a process for converting cis-1-chloro-2,3,3-trifluoro-1-propene (HCFO-1233yd(Z)) to trans-1-chloro-2,3,3-trifluoro-1-propene (HCFO-1233yd(E)) (“Process 2”).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, the phrase “light impurities” refer to molecules that have lower boiling points than the target products.

As used herein, the phrase “heavy impurities” refer to molecules that have higher boiling points than the target products.

As used herein, the “reflux ratio” is defined as the split ratio of two liquid streams from an overhead condenser of a distillation column, which may be calculated as the flow rate of the stream leaving the condenser divided by the flow rate of the stream returning to the column.

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation when substantially pure reactant is used:

$$\% conversion of molecule X = ( 100 - molecule X mol . \% in the organic components of a product mixture )$$

When the reactant includes impurities or recycled components, i.e., is a component in a reaction mixture, the conversion of a reactant molecule (molecule X) during a reaction is calculated using the following equations:

$$\% conversion of molecule X = ( change in X mol . \% ) / ( X mol \% in the reactant mixture )$$

$$OR$$

$$\% conversion of molecule X = ( X mol \% in the reactant mixture - X mol \% in the product mixture ) / ( X mol \% in the reactant mixture )$$

As used herein, selectivity to a molecule formed during a reaction (molecule X) is calculated using the following equation:

$$\% selectivity to molecule X = mol . \% of molecule X in the organic components of a product mixture / ( 100 - mol . \% of reactant molecules in the organic components of a product mixture ) \times 100.$$

When the substrate conversion rate is 100%, the mole percentages of each molecule in the resulting product mixture are equal to the selectivity of each molecule.

### II. Overview

The present disclosure provides a method for producing cis-HCFO-1233yd(Z) from trans-HCFO-1233yd(E) (“Process 1”), which includes providing a feedstock containing at least 50% HCFO-1233yd(E), vaporizing at least a portion of HCFO-1233yd(E), feeding the vaporized HCFO-1233yd(E) to a reactor, converting at least a portion of HCFO-1233yd(E) to HCFO-1233yd(Z), and separating and recovering HCFO-1233yd(Z) or a mixture of HCFO-1233yd(E) and HCFO-1233yd(Z). The reactor for Process 1 may be a fixed bed reactor including a catalyst.

A schematic equation for Process 1 is represented below:

### Process 1

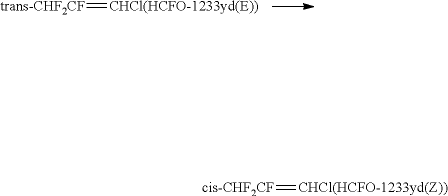

The present disclosure provides a method for producing trans-HCFO-1233yd(E) from cis-HCFO-1233yd(Z) (“Process 2”), which includes providing a feedstock containing at least 50% HCFO-1233yd(Z), vaporizing at least a portion of HCFO-1233yd(Z), feeding the vaporized HCFO-1233yd(Z) to a reactor, converting at least a portion of HCFO-1233yd(Z) to HCFO-1233yd(E), and separating and recovering HCFO-1233yd(E) or a HCFO-1233yd(E)/HCFO-1233yd(Z) mixture. The reactor for Process 2 may be an electric heater reactor.

A schematic equation for Process 2 is represented below:

### Process 2

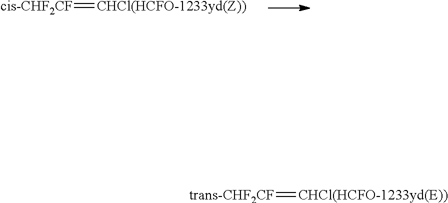

Further details regarding each of the Processes are set forth below.

### III. Process 1

### General Process

The isomerization reaction of Process 1 may be carried out in the gas vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel.

The reactor may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow may be in the down or up direction through a bed of the catalyst. Products may be flowed through one or more scrubbers to remove by-products from the reaction, such as hydrogen fluoride (HF) and/or hydrogen chloride (HCl), and the reaction products may be collected by capture in a cooled cylinder, for example.

### Process Flow 1

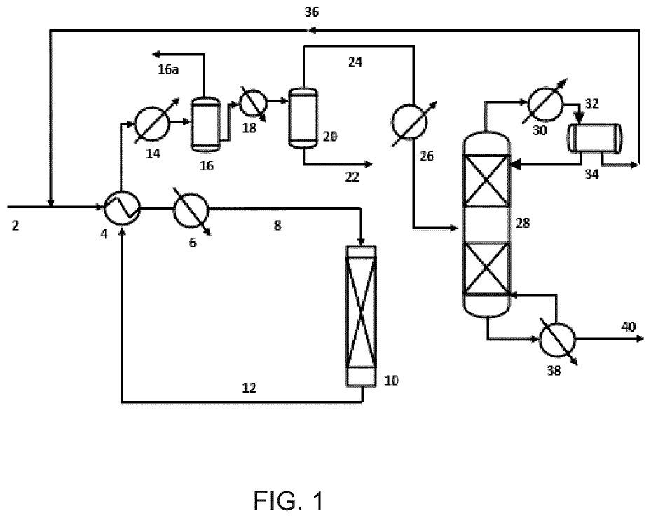

One schematic of a process flow illustrating suitable components for the reaction in Process 1 is provided in FIG. 1. FIG. 1 is a process flow diagram of a process converting trans-1-chloro-2,3,3-trifluoro-1-propene (HCFO-1233yd(E)) to cis-1-chloro-2,3,3-trifluoro-1-propene (HCFO-1233yd(Z)). The process as shown in FIG. 1 may be used in isomerization reactions described below in Examples 1, 2, and 4.

Referring to the process flow diagram shown in FIG. 1, a reactant stream 2 containing at least 50% HCFO-1233yd(E) is pumped through one or more heat exchangers 4 and 6 to vaporize at least a portion of HCFO-1233yd(E) to a temperature of from about 220° C. to about 250° C. The heat exchangers 4 and/or 6 vaporize at least about 50% of the reactant stream, or at least about 60%, or at least about 70%, or at least about 80%, or at least about 90%, or at least about 95%, or at least about 99% of the reactant stream. The reactant stream may be a liquid stream at ambient condition before entering the heat exchangers.

The heated vapor reactant stream 8 containing HCFO-1233yd(E) enters a tubular reactor 10 to convert at least a portion of HCFO-1233yd(E) to HCFO-1233yd(Z) under conditions effective for the isomerization reaction of HCFO-1233yd(E) to HCFO-1233yd(Z) (i.e., Process 1). The reactor may be a fixed bed reactor that does not include a catalyst or, alternatively, the reactor may be packed with a catalyst (e.g., a solid catalyst) to accelerate the reaction rate. The catalyst may be pre-treated (e.g., partially fluorinated). The reactor is pre-heated and operated under an isothermal condition at a temperature of from about 50° C. to about 600° C., or from about 100° C. to about 400° C., or from about 200° C. to about 350° C., or about 220° C., or within any range encompassing any two of these values as endpoints.

The effluent 12 from reactor 10 passes through one or more heat exchangers 4 and 14 to recover heat. Light impurities 16a from the stream may be removed from the vapor line of a knockout container 16 at ambient conditions (e.g., P=1 atm, T=20° C.). The bottom stream of 16 may go through another heat exchanger 18, and heavy impurities 22 are removed from the bottom liquid line of a flash drum 20 at atmospheric pressure and a temperature of from about 60° C. to about 70° C., or at about 65° C., or within any range encompassing any two of these values as endpoints.

After passing through a heat exchanger 26, the overhead stream 24 from flash drum 20 may enter a separation column 28 to separate and recover HCFO-1233yd(Z) or a mixture of HCFO-1233yd(E) and HCFO-1233yd(Z). The overhead condenser of column 28 may be cooled with cooling water and the reboiler is heated with a low-pressure steam. The overhead condenser may be operated at 1 atm and the process stream temperature is from about 40° C. to about 60° C., or from about 45° C. to about 55° C., or from about 49.7° C. to about 50° C., or within any range encompassing any two of these values as endpoints. The bottom temperature is from about 50° C. to about 70° C., or from about 55° C. to about 65° C., or from about 63.8° C. to about 64° C., or within any range encompassing any two of these values as endpoints.

The heat exchangers (e.g., 4, 14, 18, 26, 30, 36, or any of the heat exchangers discussed herein) may be an economizer or interchanger to recover heat from reactor effluent. The heat exchanger may be a shell and tube heat exchanger, for example. One or more of the heat exchangers 4, 14, 18, 26, 30, 36 may be employed to improve the energy efficiency of the process.

The process may include separating unreacted reactants, including unreacted HCFO-1233yd(E), via distillation and recycling these unreacted reactants back to the reactor 10 in FIG. 1. The distillate stream 32 from the overhead condenser of column 28 may be collected in container 34 and recycled to the front end of the reactor to mix with the feed stream via recycle stream 36. The bottom stream from 28 is the product stream 40 containing the end product of HCFO-1233yd(Z) or a mixture of HCFO-1233yd(E) and HCFO-1233yd(Z) is collected and analyzed. The bottom stream includes from about 80% to about 99.99% of 1233yd(Z) and from about 0.01% to about 20% 1233yd(E), or from about 85% to about 99.99% of 1233yd(Z) and from about 0.01% to about 15% 1233yd(E), or from about 90% to about 99.99% of 1233yd(Z) and from about 0.01% to about 10% 1233yd(E), or from about 95% to about 99.99% of 1233yd(Z) and from about 0.01% to about 5% 1233yd(E), or from about 98% to about 99.99% of 1233yd(Z) and from about 0.01% to about 2% 1233yd(E), or within any range encompassing any two of these values as endpoints, based on total moles of organic components of the composition.

### Process Flow 2

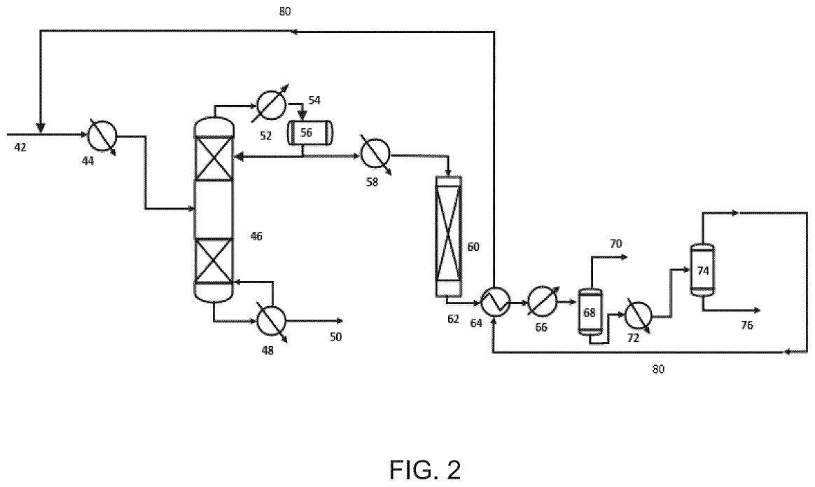

One schematic of a process flow illustrating suitable components for the reaction in Process 1 is provided in FIG. 2. FIG. 2 is a process flow diagram of a process converting trans-1-chloro-2,3,3-trifluoro-1-propene (HCFO-1233yd(E)) to cis-1-chloro-2,3,3-trifluoro-1-propene (HCFO-1233yd(Z)). The process as shown in FIG. 2 may be used in isomerization reactions described below in Example 3. A reactant stream may include a mixture of HCFO-1233yd(Z) and HCFO-1233yd(E) or a higher percentage of HCFO-1233yd(Z) than HCFO-1233yd(E), in which cases, in order to carry out Process 1, a separator (e.g., separation column 46) may be used to separate the isomers in the stream before entering the reactor (e.g., reactor 60).

Referring to the process flow diagram shown in FIG. 2, a feedstock of a reactant stream 42 containing a mixture of HCFO-1233yd(Z) and HCFO-1233yd(E) (e.g., at least 50% of HCFO-1233yd(Z)) is heated by heat exchanger 44 to its bubble point at 1 atmospheric pressure to vaporize at least a portion of the reaction stream. The heat exchangers 44 and/or 48 vaporize at least about 50% of the reactant stream, or at least about 60%, or at least about 70%, or at least about 80%, or at least about 90%, or at least about 95%, or at least about 99% of the reactant stream. The reactant stream may be a liquid stream at ambient condition before going through the heat exchangers. The reactant stream may have a bubble point of from about 40° C. to about 70° C., or from about 45° C. to about 65° C., or from about 50° C. to about 60° C., or about 54.5° C., or within any range encompassing any two of these values as endpoints.

The heated vapor reactant stream containing a mixture of HCFO-1233yd(Z) and HCFO-1233yd(E) is fed to a separation column 46 to separate and recover HCFO-1233yd(Z), or a mixture of HCFO-1233yd(E) and HCFO-1233yd(Z) with a higher percentage of HCFO-1233yd(E). Before entering the separation column 46, the reaction stream contains about 50% of HCFO-1233yd(Z) and about 50% of HCFO-1233yd(E), or about 60% HCFO-1233yd(Z) and about 40% of HCFO-1233yd(E), or about 70% HCFO-1233yd(Z) and about 30% of HCFO-1233yd(E), or about 80% HCFO-1233yd(Z) and about 20% of HCFO-1233yd(E), or about 90% HCFO-1233yd(Z) and about 10% of HCFO-1233yd(E), or about 95% of HCFO-1233yd(Z) and about 5% of HCFO-1233yd(E), or within any range encompassing any two of these values as endpoints, based on total moles of organic components of the composition. The reaction stream recovered from after going through the separation column 46 contains about 80% HCFO-1233yd(E) and about 20% of HCFO-1233yd(Z), or about 90% HCFO-1233yd(E) and about 10% of HCFO-1233yd(Z), or about 95% of HCFO-1233yd(E) and about 5% of HCFO-1233yd(Z), or within any range encompassing any two of these values as endpoints, based on total moles of organic components of the composition.

The overhead condenser of column 46 may be cooled with cooling water and the reboiler is heated with low-pressure steam. The overhead condenser may be operated at 1 atm and the process stream temperature is from about 40° C. to about 60° C., or from about 45° C. to about 55° C., or from about 49.7° C. to about 50° C., or within any range encompassing any two of these values as endpoints. The bottom temperature is from about 50° C. to about 70° C., or from about 55° C. to about 65° C., or from about 63.8° C. to about 64° C., or within any range encompassing any two of these values as endpoints.

The distillate stream 54 from the overhead condenser of column 46 may be collected in container 56 and heated up to about 220° C. before it is fed into a tubular reactor 60 to convert at least a portion of HCFO-1233yd(E) to HCFO-1233yd(Z) under conditions effective for the isomerization reaction of HCFO-1233yd(E) to HCFO-1233yd(Z) (i.e., Process 1). The distillate stream includes at least 50% 1233yd(E) (e.g., about 90% 1233yd(E) and about 10% 1233yd(Z)). The reactor may have an internal diameter of about 1″ and a length of about 120″. The reactor may be a fix-bed reactor. The reactor may be packed with a catalyst (e.g., a solid catalyst such as AlCl₃). The catalysts may be pre-treated (e.g., partially fluorinated). The reactor is pre-heated and operated under an isothermal condition at a temperature of from about 50° C. to about 600° C., or from about 100° C. to about 400° C., or from about 200° C. to about 350° C., or about 220° C., or within any range encompassing any two of these values as endpoints.

The effluent 62 from 60 may pass through one or more heat exchangers 64 and 66 to recover heat. Light impurities 70 from the stream may be removed from the vapor line of a knockout container 68 at ambient conditions (e.g., P=1 atm, T=20° C.). The bottom stream of container 68 may go through another heat exchanger 72, and heavy impurities 76 are removed from the bottom liquid line of a flash drum 74 at atmospheric pressure and a temperature of from about 60° C. to about 70° C., or at about 65° C., or within any range encompassing any two of these values as endpoints.

The heat exchangers (e.g., 44, 48, 52, 58, 64, 66, 72, or any of the heat exchangers discussed herein) may be an economizer or interchanger to recover heat from reactor effluent. The heat exchanger may be a shell and tube heat exchanger, for example. One or more of the heat exchangers 44, 48, 52, 58, 64, 66, 72 may be employed to improve the energy efficiency of the process.

The process includes separating unreacted reactants, including unreacted HCFO-1233yd(E), via distillation and recycling these unreacted reactants back to the reactor. The overhead stream 80 of flash drum 74 goes back through heat exchanger 64 and is recycled to the front end of the column to mix with the feed stream to the separation tower 46. The bottom stream 50 from 46 containing the end product of HCFO-1233yd(Z) or a mixture of HCFO-1233yd(E) and HCFO-1233yd(Z) is collected and analyzed. The bottom stream includes from about 80% to about 99.99% of 1233yd(Z) and from about 0.01% to about 20% 1233yd(E), or from about 85% to about 99.99% of 1233yd(Z) and from about 0.01% to about 15% 1233yd(E), or from about 90% to about 99.99% of 1233yd(Z) and from about 0.01% to about 10% 1233yd(E), or from about 95% to about 99.99% of 1233yd(Z) and from about 0.01% to about 5% 1233yd(E), or from about 98% to about 99.99% of 1233yd(Z) and from about 0.01% to about 2% 1233yd(E), or within any range encompassing any two of these values as endpoints, based on total moles of organic components of the composition.

Process Flow 1 is used instead of Process Flow 2 when the feedstock contains a higher percentage of 1233yd(E), for example higher than 95% (i.e., the reactant stream is of high purity). Process Flow 2 is used instead of Process Flow 1 when the feedstock contains a lower percentage of 1233yd(E), for example from about 90% to about 95% (i.e., the reactant stream is more of a mixture of 1233yd(E) and 1233yd(Z)).

### Catalysts for Process 1

The catalyst active to catalyze the reaction in Process 1 may be Chromium (Cr) based catalysts, promoted Cr-based catalysts, and/or non-Cr based catalysts.

Suitable Cr-based catalysts may include chromium oxides, chromium oxyfluorides (e.g., CrOₓF_{y} where x may be greater than 0 but less than 1.5, and y may be greater than 0 but less than 3), and/or chromium halides (CrX₂, or CrX₃, where X═F, Cl, Br, or I). As will be discussed in more detail below regarding catalyst pretreatment, the Cr-based catalysts may be fluorinated. For example, suitable Cr-based catalyst may be fluorinated Cr₂O₃.

Promoted Cr-based catalyst may be based on chromium and comprises a co-catalyst selected from the group consisting of Ni, Zn, Co, Mn, Mg, or mixtures hereof. The cocatalyst content is between 0.1% and 20% based on the total weight of the catalyst. A catalyst including zinc oxide and chromium oxide (e.g., JM 62-3M containing zin/chromia) may be used. In some instances, a catalyst including zinc oxide and chromium oxide may be ZnO—Cr₂O₃.

Non-Cr-based catalysts may be selected from the group consisting of: alumina, iron oxide, magnesium oxide, zinc oxide, nickel oxide, cobalt oxide, aluminum fluoride; iron fluoride, magnesium fluoride, zinc fluoride, nickel fluoride, cobalt fluoride, fluorinated alumina; fluorinated iron oxide, fluorinated magnesium oxide, fluorinated nickel oxide, fluorinated cobalt oxide, or mixtures thereof. Non-Cr-based catalysts may include AlCl₃. In some instances, non-Cr-based catalysts may include partially fluorinated AlCl₃, or fluorinated AlCl₃.

The catalyst may include chromium (III) oxides, such as crystalline chromium oxide or amorphous chromium oxide that are pretreated with fluorination, as discussed in more details below. While not limited thereto, amorphous chromium oxide (Cr₂O₃) may be a preferred vapor phase catalyst. It is a commercially available material in a variety of particle sizes and may be selected to enhance their effectiveness. Chromium oxide catalyst is provided having a purity of at least 98%. The fluorination catalyst is provided in any amount sufficient to drive the reaction, but also may be presented in excess.

Suitable catalyst for use in the reactor (10 or 60) for carrying out the isomerization reaction of Process 1, referring to FIGS. 1 and 2, are listed in Table 1 below.

TABLE 1
Catalysts - Process 1 Isomerization Reaction

| Catalyst | Cocatalyst |
|---|---|
| chromium oxide (Cr₂O₃) | None |
| chromium oxyfluorides (CrOₓF_{y}) | None |
| chromium halide (CrX₂ or CrX₃) | None |
| chromium oxide (Cr₂O₃) | ZnO |
| Cr₂O₃ | Ni |
| Cr₂O₃ | Zn |
| Cr₂O₃ | Co |
| Cr₂O₃ | Mn |
| Cr₂O₃ | Mg |
| fluorinated Cr₂O₃ | None |
| alumina | None |
| iron oxide | None |
| magnesium oxide | None |
| zinc oxide | None |
| nickel oxide | None |
| cobalt oxide | None |
| aluminum fluoride | None |
| iron fluoride | None |
| magnesium fluoride | None |
| zinc fluoride | None |
| nickel fluoride | None |
| cobalt fluoride | None |
| fluorinated alumina | None |
| fluorinated iron oxide | None |
| fluorinated magnesium oxide | None |
| fluorinated nickel oxide | None |
| fluorinated cobalt oxide | None |
| aluminum chloride (AlCl₃) | None |
| (partially) fluorinated AlCl₃ | None |

### Process 1—Catalyst BET Surface Area

The catalyst used in Process 1 may have a proper BET (Brunauer, Emmet, and Teller) surface area. The BET surface area of the catalyst may be as low as about 1 m²/g, about 3 m²/g, about 5 m²/g, about 10 m²/g, about 15 m²/g, about 20 m²/g², about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, or within any range encompassed by any of the foregoing values as endpoints. Specific examples of additional suitable ranges are set forth below in Table 2. The numerical ranges set forth in Table 2 below are understood to be prefaced by “about”.

The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

TABLE 2
BET Surface Area of Catalysts - Process 1 Isomerization Reaction

| From (m²/g) | To (m²/g) |
|---|---|
| 1 | 1000 |
| 1 | 500 |
| 1 | 200 |
| 1 | 100 |
| 1 | 20 |
| 5 | 1000 |
| 5 | 500 |
| 5 | 200 |
| 5 | 100 |
| 5 | 20 |
| 10 | 1000 |
| 10 | 500 |
| 10 | 200 |
| 10 | 100 |
| 10 | 20 |
| 1 | 3 |
| 3 | 5 |
| 5 | 10 |
| 10 | 15 |

### Process 1—Catalyst Pretreatment

The catalyst used in Process 1 may be pretreated by a variety of methods to improve its performance and effectiveness in the reaction. For example, the catalyst may be dried at elevated temperatures, as low as about 100° C., 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints. Specific examples of additional suitable ranges are set forth below in Table 3. The numerical ranges set forth in Table 3 below are understood to be prefaced by “about”.

TABLE 3
Pre-treatment Drying Temperatures for Catalysts
in Table 1 - Process 1 Isomerization Reaction

| From (° C.) | To (° C.) |
|---|---|
| 100 | 400 |
| 120 | 400 |
| 150 | 400 |
| 200 | 400 |
| 100 | 400 |
| 100 | 350 |
| 100 | 300 |
| 100 | 250 |
| 100 | 200 |
| 100 | 150 |
| 100 | 120 |
| 120 | 200 |
| 200 | 300 |
| 300 | 400 |

As part of the catalyst pretreatment for catalysts used in Process 1 as listed in Table 1, the catalyst may be exposed to an inert gas such as N₂. The pretreatment process may take as low as about 1 hour, about 2 hours, about 3 hours, or as high as about 4 hours, about 5 hours, about 6 hours, about 10 hours, about 20 hours, about 1 day, about 2 days, about 5 days, about 10 days, about 20 days, or within any range encompassed by two of the foregoing values as endpoints such as about 2 hours to about 4 hours, for example.

When a Cr-based or promoted Cr-based catalyst is used, a fluorination treatment of said catalyst may be conducted using anhydrous HF under conditions effective to convert a portion of metal oxides into corresponding metal fluorides.

When a non-Cr-based catalyst is used, a fluorination treatment of catalysts containing metal oxide(s) may be conducted using anhydrous HF under conditions effective to convert a portion of metal oxide(s) (e.g., zinc oxide, magnesium oxide, aluminum oxide) into corresponding metal fluoride(s).

Pre-treatment such as fluorination treatment may help prevent the raw material containing HCFO-1233yd(Z) or HCFO-1233yd(E) from reacting with the catalyst to form undesired byproducts. When catalysts are pre-treated with fluorination, little or no further fluorination reaction will happen to the catalysts during reaction in reactor R01, thus making the reaction more selective.

### Process 1 Reaction Condition—Temperature

As Process 1 is an exothermic reaction, the selectivity towards the desired product HCFO-1233yd(Z) may increase as temperature decreases, whereas the selectivity towards HCFO-1233yd(E) increases with temperature. The reaction temperature for Process 1 may be as low as about 50° C., about 100° C., about 150° C., about 200° C., about 250° C. or as high as about 350° C., about 400° C., about 450° C., about 500° C., about 550° C., about 600° C., or within any range encompassed by two of the foregoing values as endpoints, such as from about 50° C. to about 600° C., or from about 100° C. to about 400° C., for example. The temperature may be preferably from about 200° C. to about 350° C., and more preferably from about 220° C. to about 250° C. Specific examples of additional suitable ranges are set forth below in Table 4. The numerical ranges set forth in Table 4 below are understood to be prefaced by “about”.

TABLE 4
Reaction Temperature when using Catalysts in
Table 1 - Process 1 Isomerization Reaction

| From (° C.) | To (° C.) |
|---|---|
| 50 | 600 |
| 50 | 500 |
| 50 | 400 |
| 50 | 350 |
| 50 | 300 |
| 50 | 250 |
| 100 | 600 |
| 100 | 500 |
| 100 | 400 |
| 100 | 350 |
| 100 | 300 |
| 100 | 250 |
| 50 | 100 |
| 100 | 150 |
| 150 | 200 |
| 200 | 220 |
| 220 | 250 |

### Process 1 Reaction Condition—Contact Time

The contact time of the reactants with each of the catalyst listed in Table 1 for Process 1 may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be preferably from about 0.1 second to about 60 seconds. Specific examples of additional suitable ranges are set forth below in Table 5. The numerical ranges set forth in Table 5 below are understood to be prefaced by “about”.

TABLE 5
Contact Time when using Catalysts in Table
1 - Process 1 Isomerization Reaction

| From (seconds) | To (seconds) |
|---|---|
| 0.1 | 120 |
| 0.1 | 100 |
| 0.1 | 80 |
| 0.1 | 60 |
| 1 | 120 |
| 1 | 100 |
| 1 | 80 |
| 1 | 60 |
| 0.1 | 1 |
| 1 | 10 |
| 10 | 20 |
| 20 | 30 |
| 30 | 40 |
| 40 | 50 |
| 50 | 60 |
| 60 | 80 |
| 80 | 100 |
| 100 | 120 |

### Process 1 Reaction Condition—Pressure

The pressure within the reactor for Process 1 may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints. For example, the pressure may be preferably from about 10 psig to about 100 psig. Specific examples of additional suitable ranges are set forth below in Table 6. The numerical ranges set forth in Table 6 below are understood to be prefaced by “about”.

TABLE 6
Pressure within reactor when using Catalysts
in Table 1 - Process 1 Isomerization Reaction

| From (psig) | To (psig) |
|---|---|
| 1 | 300 |
| 1 | 250 |
| 1 | 200 |
| 1 | 150 |
| 1 | 100 |
| 10 | 300 |
| 10 | 250 |
| 10 | 200 |
| 10 | 150 |
| 10 | 100 |
| 1 | 10 |
| 10 | 50 |
| 50 | 100 |
| 100 | 150 |
| 150 | 200 |
| 200 | 250 |
| 250 | 300 |

A summary of the preferred catalyst, contact time, temperature, and pressure as discussed above are summarized in Table 7 below. The numerical ranges set forth in Table 7 below are understood to be prefaced by “about”.

TABLE 7
Summary of Catalyst and Reaction Conditions for Process 1

| | Contact | Pressure | Temperature |
|---|---|---|---|
| Catalyst | Time (s) | (psig) | (° C.) |
| fluorinated AlCl₃ | 0.1-120 | 1-300 | 100-400 |
| fluorinated AlCl₃ | 0.1-120 | 1-300 | 200-350 |
| fluorinated AlCl₃ | 0.1-120 | 10-100 | 100-400 |
| fluorinated AlCl₃ | 0.1-120 | 10-100 | 200-350 |
| fluorinated AlCl₃ | 1-60 | 1-300 | 100-400 |
| fluorinated AlCl₃ | 1-60 | 1-300 | 200-350 |
| fluorinated AlCl₃ | 1-60 | 10-100 | 100-400 |
| fluorinated AlCl₃ | 1-60 | 10-100 | 200-350 |
| fluorinated Cr₂O₃ | 0.1-120 | 1-300 | 100-400 |
| fluorinated Cr₂O₃ | 0.1-120 | 1-300 | 200-350 |
| fluorinated Cr₂O₃ | 0.1-120 | 10-100 | 100-400 |
| fluorinated Cr₂O₃ | 0.1-120 | 10-100 | 200-350 |
| fluorinated Cr₂O₃ | 1-60 | 1-300 | 100-400 |
| fluorinated Cr₂O₃ | 1-60 | 1-300 | 200-350 |
| fluorinated Cr₂O₃ | 1-60 | 10-100 | 100-400 |
| fluorinated Cr₂O₃ | 1-60 | 10-100 | 200-350 |
| ZnO—Cr₂O₃ | 0.1-120 | 1-300 | 100-400 |
| ZnO—Cr₂O₃ | 0.1-120 | 1-300 | 200-350 |
| ZnO—Cr₂O₃ | 0.1-120 | 10-100 | 100-400 |
| ZnO—Cr₂O₃ | 0.1-120 | 10-100 | 200-350 |
| ZnO—Cr₂O₃ | 1-60 | 1-300 | 100-400 |
| ZnO—Cr₂O₃ | 1-60 | 1-300 | 200-350 |
| ZnO—Cr₂O₃ | 1-60 | 10-100 | 100-400 |
| ZnO—Cr₂O₃ | 1-60 | 10-100 | 200-350 |
| None | 0.1-120 | 1-300 | 100-400 |
| None | 0.1-120 | 1-300 | 200-350 |
| None | 0.1-120 | 10-100 | 100-400 |
| None | 0.1-120 | 10-100 | 200-350 |
| None | 1-60 | 1-300 | 100-400 |
| None | 1-60 | 1-300 | 200-350 |
| None | 1-60 | 10-100 | 100-400 |
| None | 1-60 | 10-100 | 200-350 |

### Process 1 Products—Selectivity

As demonstrated by the Examples herein, Process 1 may achieve a selectivity to the HCFO-1233yd(Z) product of greater than about 20%, greater than about 30%, greater than about 40%, greater than about 50%, greater than about 60%, greater than about 70%, greater than about 80%, greater than about 85%, greater than about 90%, greater than about 95%, greater than about 98%, greater than about 99%, greater than about 99.9%, greater than about 99.99%, and for each of the foregoing, less than or equal to 100%, or within any range encompassed by two of the foregoing values as endpoints, based on total moles of organic components of the composition. Specific examples of additional suitable ranges are set forth below in Table 8. The numerical ranges set forth in Table 8 below are understood to be prefaced by “about”.

TABLE 8
Selectivity to HCFO-1233yd(Z) in Process 1

| From (%) | To (%) |
|---|---|
| 20 | ≤100 |
| 30 | ≤100 |
| 40 | ≤100 |
| 50 | ≤100 |
| 60 | ≤100 |
| 70 | ≤100 |
| 80 | ≤100 |
| 90 | ≤100 |
| 95 | ≤100 |
| 98 | ≤100 |
| 99 | ≤100 |
| 99.9 | ≤100 |
| 99.99 | ≤100 |
| 20 | 40 |
| 40 | 60 |
| 60 | 80 |
| 80 | 90 |
| 90 | 95 |
| 95 | 98 |
| 98 | 99 |
| 99 | 99.9 |
| 99.9 | 99.99 |

### Process 1 Products—Conversion

As also demonstrated by the Examples, herein, Process 1 may achieve a conversion of HCFO-1233yd(E) to HCFO-1233yd(Z) of greater than about 10%, greater than about 20%, greater than about 30%, greater than about 40%, greater than about 50% greater than about 60%, greater than about 75%, greater than about 90%, greater than about 95%, greater than about 97% or greater, and for each of the foregoing, less than or equal to 100%, or within any range encompassed by two of the foregoing values as endpoints, based on total moles of organic components of the composition. For example, process 1 may achieve a conversion of HCFO-1233yd(E) to HCFO-1233yd(Z) of from about 90% to about 100%, from about 95% to about 100%, from about 97% to about 100%, or within any range encompassed by two of the foregoing values as endpoints, based on total moles of organic components of the composition. Specific examples of additional suitable ranges are set forth below in Table 9. The numerical ranges set forth in Table 9 below are understood to be prefaced by “about”.

TABLE 9
Conversion of HCFO-1233yd(E) in Process 1

| From (%) | To (%) |
|---|---|
| 10 | ≤100 |
| 20 | ≤100 |
| 30 | ≤100 |
| 40 | ≤100 |
| 50 | ≤100 |
| 60 | ≤100 |
| 70 | ≤100 |
| 75 | ≤100 |
| 80 | ≤100 |
| 90 | ≤100 |
| 95 | ≤100 |
| 97 | ≤100 |
| 98 | ≤100 |
| 99 | ≤100 |
| 99.9 | ≤100 |
| 90 | 95 |
| 95 | 98 |
| 98 | 99 |
| 99 | 99.9 |

The end product may include a mixture of HCFO-1233yd(Z) and HCFO-1233yd(E), for example, from about 80% to about 99.99% of 1233yd(Z) and from about 0.01% to about 20% 1233yd(E), or from about 85% to about 99.99% of 1233yd(Z) and from about 0.01% to about 15% 1233yd(E), or from about 90% to about 99.99% of 1233yd(Z) and from about 0.01% to about 10% 1233yd(E), or from about 95% to about 99.99% of 1233yd(Z) and from about 0.01% to about 5% 1233yd(E), or from about 98% to about 99.99% of 1233yd(Z) and from about 0.01% to about 2% 1233yd(E), or within any range encompassing any two of these values as endpoints.

Byproducts may also be produced during Process 1, for example, CHF₂—C≡CCl and CHF₂—CF═CH—CCl═CF—CHF₂, etc. These byproducts are undesirable as they are difficult to recycle or convert to useful intermediates or the end product.

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrofluorination treatment at temperatures of from about 100° C. to about 400° C., or from about 200° C. to about 350° C.

### IV. Process 2

### General Process

The isomerization reaction of Process 2 may be carried out in the gas vapor phase. Process 2 may be carried out in a tubular reactor with an external heating system such as a molten salt heating system to heat the reactor tubes to a desired inner surface temperature effective for the reaction to take place. The reactor tubes are constructed from a material which is resistant to temperature and/or corrosive effects of HF or HCl. Suitable materials include, for example, nickel and its alloys, including Hastelloy (for example, Hastelloy C276), Inconel (for example, Inconel 600), Incoloy, and Monel.

Process 2 may be carried out in an electric heater reactor with electric heater elements including a tube (e.g., a sheath), a metal alloy wire such as Nichrome wire, and a compacted metal oxide material (e.g., MgO) to heat the reactor tube to a desired outer surface temperature effective for the reaction to take place. The electric heater elements are packed with MgO powder to conduct heat from Nichrome wire while also acting as an electrical insulator. The manufacturing process includes inserting Nichrome wire into a tube (e.g., a sheath), packing with MgO powder, and rolling the tube again to compact the MgO and form a ceramic-like material. The MgO powder is isolated from the process because of the sheath tube wall throughout the interior of the heater. The sheath tubes may be constructed from a material which is resistant to temperature and/or corrosive effects of HF or HCl. Suitable materials include, for example, nickel and its alloys, including Hastelloy (for example, Hastelloy C276), Inconel (for example, Inconel 600), Incoloy, and Monel.

The reactor may be first cleaned and flushed with an inert gas such as nitrogen. The reactor does not include a catalyst.

After reaction, products may be flowed through one or more scrubbers to remove by-products from the reaction, such as hydrogen fluoride (HF) and/or hydrogen chloride (HCl), and the reaction products may be collected by capture in a cooled cylinder, for example.

### Process Flow 3

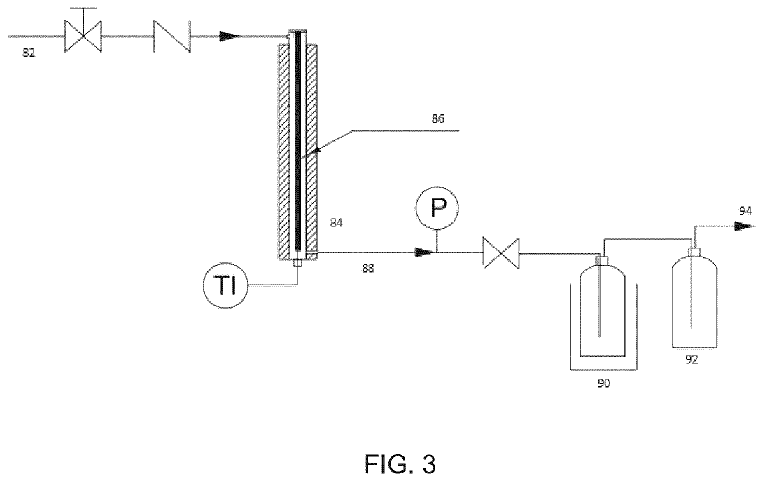

One schematic of a process flow illustrating suitable components for the reaction in Process 2 is provided in FIG. 3. FIG. 3 is a process flow diagram of a process converting cis-1-chloro-2,3,3-trifluoro-1-propene (HCFO-1233yd(Z)) to trans-1-chloro-2,3,3-trifluoro-1-propene (HCFO-1233yd(E)). The process as shown in FIG. 3 may be used in isomerization reactions described below in Example 5.

Referring to the process flow diagram shown in FIG. 3, a feedstock of a reactant stream 82 containing at least 50% HCFO-1233yd(Z) (e.g., 92% 1233yd(Z) and 8% 1233yd(E)) is pumped into an electric heater reactor 84 with an immersion heating element 86. The heating element may be configured to vaporize at least a portion of HCFO-1233yd(Z) to a temperature of about 380° C. The heating element vaporizes at least about 50% of the reactant stream, or at least about 60%, or at least about 70%, or at least about 80%, or at least about 90%, or at least about 95%, or at least about 99% of the reactant stream. The reactant stream may be a liquid stream at ambient condition before going through the reactor.

The heated vapor reactant stream containing HCFO-1233yd(Z) is fed into the electric heater reactor 84 to convert at least a portion of HCFO-1233yd(Z) to HCFO-1233yd(E) under conditions effective for the isomerization reaction of HCFO-1233yd(Z) to HCFO-1233yd(E) (i.e., Process 2). The reactor may have an internal diameter of about 1″ and a length of about 72″. The reactor may have a thermal couple on the end of the heating element for measuring the temperature. The feedstock enters from the top of the reactor 84 and flows out from the bottom of the reactor 84.

The effluent 88 from 84 flows through a dry ice trap 90 and a caustic scrubber 92 before being collected as the desired reactor product 94. After a single pass through the electric heater reactor 84 with a feedstock reactant stream containing at least 50% HCFO-1233yd(Z) (e.g., greater than 90% 1233yd(Z) and less than 10% 1233yd(E)), the product fractions of the reactor product may include a mixture of HCFO-1233yd(Z) and HCFO-1233yd(E), for example, from about 80% to about 90% of 1233yd(Z) and from about 10% to about 20% 1233yd(E), or from about 75% to about 85% of 1233yd(Z) and from about 15% to about 25% 1233yd(E), or from about 70% to about 80% of 1233yd(Z) and from about 20% to about 30% 1233yd(E), or from about 65% to about 75% of 1233yd(Z) and from about 25% to about 35% 1233yd(E), or from about 60% to about 70% of 1233yd(Z) and from about 30% to about 40% 1233yd(E), or within any range encompassing any two of these values as endpoints, based on total moles of organic components of the composition. A single pass through the electric heater reactor may achieve a conversion of HCFO-1233yd(Z) to HCFO-1233yd(E) of greater than about 17%, or greater than about 20%, or greater than about 22%, or greater than about 24%, or within any range encompassing any two of these values as endpoints, based on total moles of organic components of the composition.

The process may be repeated until a desired end product is achieved. The product stream containing the end product of HCFO-1233yd(E) or a mixture of HCFO-1233yd(E) and HCFO-1233yd(Z), is collected and analyzed. The end product stream includes from about 80% to about 99.99% of 1233yd(E) and from about 0.01% to about 20% 1233yd(Z), or from about 85% to about 99.99% of 1233yd(E) and from about 0.01% to about 15% 1233yd(Z), or from about 90% to about 99.99% of 1233yd(E) and from about 0.01% to about 10% 1233yd(Z), or from about 95% to about 99.99% of 1233yd(E) and from about 0.01% to about 5% 1233yd(Z), or from about 98% to about 99.99% of 1233yd(E) and from about 0.01% to about 2% 1233yd(Z), or within any range encompassing any two of these values as endpoints, based on total moles of organic components of the composition.

### Process Flow 4

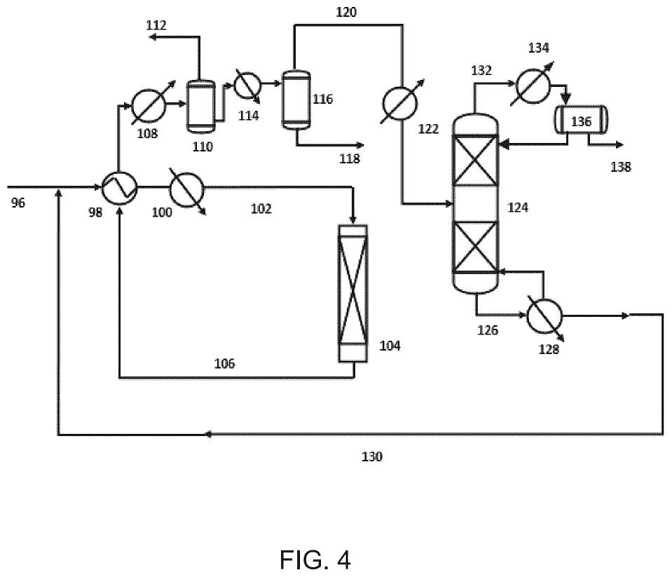

One schematic of a process flow illustrating suitable components for the reaction in Process 2 is provided in FIG. 4. FIG. 4 is a process flow diagram of a process converting cis-1-chloro-2,3,3-trifluoro-1-propene (HCFO-1233yd(Z)) to trans-1-chloro-2,3,3-trifluoro-1-propene (HCFO-1233yd(E)). The process as shown in FIG. 4 may be used in isomerization reactions described below in Example 6.

Referring to the process flow diagram shown in FIG. 4, a feedstock of a reactant stream 96 containing at least 50% HCFO-1233yd(Z) (e.g., 92% 1233yd(Z) and 8% 1233yd(E)) is conveyed through one or more heat exchangers 98 and 100 to vaporize at least a portion of HCFO-1233yd(Z) to a temperature of about 380° C. The heat exchangers 98 and 100 vaporizes at least about 50% of the reactant stream, or at least about 60%, or at least about 70%, or at least about 80%, or at least about 90%, or at least about 95%, or at least about 99% of the reactant stream. The reactant stream may be a liquid stream at ambient condition before going through the heat exchangers.

The heated vapor reactant stream 102 containing HCFO-1233yd(Z) is fed into a tubular reactor 104 to convert at least a portion of HCFO-1233yd(Z) to HCFO-1233yd(E) under conditions effective for the isomerization reaction of HCFO-1233yd(Z) to HCFO-1233yd(E) (i.e., Process 2). The reactor may have an internal diameter of about 1″ and a length of from about 40″ to about 60″. The reactor is pre-heated and operated under an isothermal condition at a temperature of from about 200° C. to about 800° C., or from about 250° C. to about 700° C., or from about 300° C. to about 600° C., or from about 350° C. to about 420° C., or within any range encompassing any two of these values as endpoints.

The effluent 106 from reactor 104 as shown in FIG. 4 passes through one or more heat exchangers 98 and 108 to recover heat. Light impurities 112 from the stream may be removed from the vapor line of a knockout container 110 at ambient conditions (e.g., P=1 atm, T=20° C.). The bottom stream of 110 may go through another heat exchanger 114, and heavy impurities 118 are removed from the bottom liquid line of a flash drum 116 at 1 atmospheric pressure and a temperature of from about 60° C. to about 70° C., or at about 65° C., or within any range encompassing any two of these values as endpoints.

The process includes separating unreacted reactants, including unreacted HCFO-1233yd(Z), via distillation and recycling these unreacted reactants back to the reactor. After going through a heat exchanger 122, the overhead stream 120 from 116 may enter a separation column 124 to separate and recover HCFO-1233yd(E) or a mixture of HCFO-1233yd(E) and HCFO-1233yd(Z). The overhead condenser of column 124 may be cooled with cooling water and the reboiler may be heated with a low-pressure steam. The overhead condenser may be operated at 1 atm and the process stream temperature is from about 40° C. to about 60° C., or at about 50° C., or within any range encompassing any two of these values as endpoints. The bottom temperature is from about 50° C. to about 70° C., or from about 55° C. to about 65° C., or from about 60° C. to about 64° C., or at about 63.8° C., or within any range encompassing any two of these values as endpoints.

The heat exchangers (e.g., 98, 100, 108, 114, 122, 128, 134, or any of the heat exchangers discussed herein) may be an economizer or interchanger to recover heat from reactor effluent. The heat exchanger may be a shell and tube heat exchanger, for example. One or more of the heat exchangers 98, 100, 108, 114, 122, 128, 134 may be employed to improve the energy efficiency of the process.

The bottom stream of column 124 goes through another heat exchanger 128 before being recycled to the front end of the reactor to mix with the feed stream via recycle stream 130. The distillate stream 132 from the overhead condenser of column 124, which is the product stream 138 containing the end product of HCFO-1233yd(E) or a mixture of HCFO-1233yd(E) and HCFO-1233yd(Z), is collected and analyzed. The distillate stream includes from about 80% to about 99.99% of 1233yd(E) and from about 0.01% to about 20% 1233yd(Z), or from about 85% to about 99.99% of 1233yd(E) and from about 0.01% to about 15% 1233yd(Z), or from about 90% to about 99.99% of 1233yd(E) and from about 0.01% to about 10% 1233yd(Z), or from about 95% to about 99.99% of 1233yd(E) and from about 0.01% to about 5% 1233yd(Z), or from about 98% to about 99.99% of 1233yd(E) and from about 0.01% to about 2% 1233yd(Z), or within any range encompassing any two of these values as endpoints, based on total moles of organic components of the composition.

### Process 2 Reaction Condition—Temperature

As Process 1 is an endothermic reaction, the selectivity towards the desired product HCFO-1233yd(E) may increase with the temperature, whereas the selectivity towards HCFO-1233yd(Z) decreases as temperature increases. The reaction temperature may be as low as about 200° C., about 250° C., about 300° C., about 350° C., about 400° C. or as high as about 420° C., about 450° C., about 500° C., about 600° C., about 700° C., about 800° C., or within any range encompassed by two of the foregoing values as endpoints, such as from about 200° C. to about 800° C., or from about 250° C. to about 700° C., for example. The temperature may be preferably from about 300° C. to about 600° C., and more preferably from about 350° C. to about 420° C. Specific examples of additional suitable ranges are set forth below in Table 10. The numerical ranges set forth in Table 10 below are understood to be prefaced by “about”.

TABLE 10
Reaction Temperature of Process 2

| From (° C.) | To (° C.) |
|---|---|
| 200 | 800 |
| 250 | 800 |
| 300 | 800 |
| 350 | 800 |
| 400 | 800 |
| 200 | 700 |
| 200 | 600 |
| 200 | 500 |
| 200 | 450 |
| 200 | 420 |
| 200 | 250 |
| 250 | 300 |
| 300 | 350 |
| 350 | 400 |
| 400 | 420 |
| 420 | 450 |
| 450 | 500 |
| 500 | 600 |
| 600 | 700 |
| 700 | 800 |

### Process 2 Reaction Condition—Pressure

The pressure may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints. For example, the pressure may be preferably from about 10 psig to about 100 psig. Specific examples of additional suitable ranges are set forth below in Table 11. The numerical ranges set forth in Table 11 below are understood to be prefaced by “about”.

TABLE 11
Reaction Pressure of Process 2

| From (psig) | To (psig) |
|---|---|
| 1 | 300 |
| 1 | 250 |
| 1 | 200 |
| 1 | 150 |
| 1 | 100 |
| 10 | 300 |
| 10 | 250 |
| 10 | 200 |
| 10 | 150 |
| 10 | 100 |
| 1 | 10 |
| 10 | 50 |
| 50 | 100 |
| 100 | 150 |
| 150 | 200 |
| 200 | 250 |
| 250 | 300 |

A summary of the preferred catalyst, contact time, temperature, and pressure as discussed above are summarized in Table 12 below. The numerical ranges set forth in Table 12 below are understood to be prefaced by “about”.

TABLE 12
Summary of Reaction Conditions for Process 2

| Catalyst | Reactor Type | Pressure (psig) | Temperature (° C.) |
|---|---|---|---|
| None | Electric heater | 1-300 | 200-700 |
| None | Electric heater | 1-300 | 300-600 |
| None | Electric heater | 10-100 | 200-700 |
| None | Electric heater | 10-100 | 300-600 |
| None | Tubular | 1-300 | 250-800 |
| None | Tubular | 1-300 | 350-700 |
| None | Tubular | 10-100 | 250-800 |
| None | Tubular | 10-100 | 350-700 |

### Process 2 Products—Selectivity

As demonstrated by the Examples herein, Process 2 (e.g., carried out through, for example, Process Flow 3 or Process Flow 4 as described herein) may achieve a selectivity to the HCFO-1233yd(E) product of greater than about 20%, greater than about 30%, greater than about 40%, greater than about 50%, greater than about 60%, greater than about 70%, greater than about 80%, greater than about 85%, greater than about 90%, greater than about 95%, greater than about 98%, greater than about 99%, greater than about 99.9%, greater than about 99.99%, and for each of the foregoing, less than or equal to 100%, or within any range encompassed by two of the foregoing values as endpoints, based on total moles of organic components of the composition. Specific examples of additional suitable ranges are set forth below in Table 13. The numerical ranges set forth in Table 13 below are understood to be prefaced by “about”.

TABLE 13
Selectivity to HCFO-1233yd(E) in Process 2

| From (%) | To (%) |
|---|---|
| 20 | ≤100 |
| 30 | ≤100 |
| 40 | ≤100 |
| 50 | ≤100 |
| 60 | ≤100 |
| 70 | ≤100 |
| 80 | ≤100 |
| 90 | ≤100 |
| 95 | ≤100 |
| 98 | ≤100 |
| 99 | ≤100 |
| 99.9 | ≤100 |
| 99.99 | ≤100 |
| 20 | 40 |
| 40 | 60 |
| 60 | 80 |
| 80 | 90 |
| 90 | 95 |
| 95 | 98 |
| 98 | 99 |
| 99 | 99.9 |
| 99.9 | 99.99 |

### Process 2 Products—Conversion

As also demonstrated by the Examples, herein, Process 2 may achieve a conversion of HCFO-1233yd(Z) to HCFO-1233yd(E) of greater than about 10%, greater than about 20%, greater than about 30%, greater than about 40%, greater than about 50% greater than about 60%, greater than about 75%, greater than about 90%, greater than about 95%, greater than about 97% or greater, and for each of the foregoing, less than or equal to 100%, or within any range encompassed by two of the foregoing values as endpoints, based on total moles of organic components of the composition. Process 2 may achieve a conversion of HCFO-1233yd(Z) to HCFO-1233yd(E) of greater than about 90%, greater than about 95%, greater than about 97%, and for each of the foregoing, less than or equal to 100%, or within any range encompassed by two of the foregoing values as endpoints, based on total moles of organic components of the composition. Specific examples of additional suitable ranges are set forth below in Table 14. The numerical ranges set forth in Table 14 below are understood to be prefaced by “about”.

TABLE 14
Conversion of HCFO-1233yd(Z) in Process 2

| From (%) | To (%) |
|---|---|
| 10 | ≤100 |
| 20 | ≤100 |
| 30 | ≤100 |
| 40 | ≤100 |
| 50 | ≤100 |
| 60 | ≤100 |
| 70 | ≤100 |
| 75 | ≤100 |
| 80 | ≤100 |
| 90 | ≤100 |
| 95 | ≤100 |
| 97 | ≤100 |
| 98 | ≤100 |
| 99 | ≤100 |
| 99.9 | ≤100 |
| 90 | 95 |
| 95 | 98 |
| 98 | 99 |

The end product may include a mixture of HCFO-1233yd(E) and HCFO-1233yd(Z), for example, from about 80% to about 99.99% of 1233yd(E) and from about 0.01% to about 20% 1233yd(Z), or from about 85% to about 99.99% of 1233yd(E) and from about 0.01% to about 15% 1233yd(Z), or from about 90% to about 99.99% of 1233yd(E) and from about 0.01% to about 10% 1233yd(Z), or from about 95% to about 99.99% of 1233yd(E) and from about 0.01% to about 5% 1233yd(Z), or from about 98% to about 99.99% of 1233yd(E) and from about 0.01% to about 2% 1233yd(Z), or within any range encompassing any two of these values as endpoints, based on total moles of organic components of the composition.

Byproducts may also be produced during Process 2, for example, CHF₂—C≡CCl and CHF₂—CF═CH—CCl═CF—CHF₂, etc. These byproducts are undesirable as they are difficult to recycle or convert to useful intermediates or the end product.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### EXAMPLES

### Example 1—Isomerization of 1233yd(E) to 1233yd(Z)

The process flow diagram is shown in FIG. 1. A liquid stream of 99.9% 1233yd(E) and 0.1% 1233yd(Z) (ambient condition) is pumped (flow rate: 1.3 kg/hr) through a series of heat exchangers to T=220° C. The heated vapor stream is fed into a tubular reactor (10, 1″(ID)×158″) which was packed with partially fluorinated AlCl₃. The reactor is pre-heated and operated under an isothermal condition (T=220° C.). The effluent from 10 passes through heat exchangers to recover heat. Light impurities 16a are removed from the vapor line of a knockout container (16, P=1 atm, T=20° C.). Heavy impurities 22 are removed from the bottom liquid line of a flash drum (20, P=1 atm, T=65° C.). The overhead stream from 20 enters the separation column (28), which has about 80 theoretical stages, and a vapor-liquid equilibrium may be achieved at a theoretical stage. The overhead condenser is cooled with cooling water and the reboiler is heated with LP steam. The reflux ratio is set to 30. The overhead condenser is operated at 1 atm and the process stream temperature is around 49.7° C. The bottom temperature is about 63.8° C. The distillate is mixed with the feed stream to the front end of the reactor. The bottom stream 40 from 28 is collected and analyzed to be 99.3% 1233yd(Z) and 0.7% 1233yd(E).

The above isomerization process is repeated with fluorinated Cr₂O₃ catalyst and fluorinated ZnO—Cr₂O₃ catalyst, respectively, and similar results are obtained.

### Example 2—Isomerization of 1233yd(E) to 1233yd(Z)

The process flow diagram is shown in FIG. 1. A liquid stream of 92% 1233yd(E) and 8% 1233yd(Z) (ambient condition) is pumped (flow rate: 1.3 kg/hr) through a series of heat exchangers to T=220° C. The heated vapor stream is fed into a tubular reactor (10, 1″(ID)×158″) which was packed with partially fluorinated AlCl₃. The reactor is pre-heated and operated under an isothermal condition (T=220° C.). The effluent from 10 passes through heat exchangers to recover heat. Light impurities are removed from the vapor line of a knockout container (16, P=1 atm, T=20° C.). Heavy impurities 22 are removed from the bottom liquid line of a flash drum (20, P=1 atm, T=65° C.). The overhead stream from 20 enters the separation column (28), which has about 80 theoretical stages, and a vapor-liquid equilibrium may be achieved at a theoretical stage. The overhead condenser is cooled with cooling water and the reboiler is heated with LP steam. The reflux ratio is set to 30. The overhead condenser is operated at 1 atm and the process stream temperature is around 49.7° C. The bottom temperature is about 63.8° C. The distillate is mixed with the feed stream to the front end of the reactor. The bottom stream 40 from 28 is collected and analyzed to be 99.3% 1233yd(Z) and 0.7% 1233yd(E).

The above isomerization process is repeated with fluorinated Cr₂O₃ catalyst and fluorinated ZnO—Cr₂O₃ catalyst, respectively, and similar results are obtained.

### Example 3—Isomerization of 1233yd(E) to 1233yd(Z)

The process flow diagram is shown in FIG. 2. A liquid stream of 9% 1233yd(E) and 91% 1233yd(Z) is heated to its bubble point (P=1 atm, T=54.5° C.) and then fed (flow rate: 1.3 kg/hr) to a separation column (46), which has about 80 theoretical stages, and a vapor-liquid equilibrium may be achieved at a theoretical stage. The overhead condenser is cooled with cooling water and the reboiler is heated with LP steam. The overhead condenser is operated at 1 atm and the process stream temperature is around 49.7° C. The bottom temperature is about 63.8° C. The reflux ratio is set to 30. The distillate steam (90% 1233yd(E) and 10% 1233yd(Z)) is heated up to T=220° C. before it is fed into a tubular reactor (60, 1″(ID)×120″(L)) which was packed with partially fluorinated AlCl₃. The effluent from 60 passes through a heat exchanger to recover heat. Light impurities 70 are removed from the vapor line of a knockout container (68, P=1 atm, T=20° C.). The liquid stream from 68 is fed into another flash drum (74, P=1 atm, T=65° C.) to remove the heavy impurities 76 from the bottom. The overhead stream of 74 is mixed with the feed steam to the separation tower (46). The bottom stream of 46 is collected as the product stream 50 which has 99.8% 1233yd(Z) and 0.2% 1233yd(E).

The above isomerization process is repeated with fluorinated Cr₂O₃ catalyst and fluorinated ZnO—Cr₂O₃ catalyst, respectively, and similar results are obtained.

### Example 4—Isomerization of 1233yd(E) to 1233yd(Z)

The process flow diagram is shown in FIG. 1. A stream of 99.9% 1233yd (E) and 0.1% 1233yd(Z) is pumped (flow rate: 1.3 kg/hr) through a series of heat exchangers to T=250° C. The heated stream flows into a tubular reactor (10, 1″(ID)×315″(L)). The reactor is pre-heated and operated under an isothermal condition (T=250° C.). The effluent from 10 passes through heat exchangers to recover heat. Light impurities are removed from the vapor line of a knockout container (16, P=1 atm, T=20° C.). Heavy impurities are removed from the bottom liquid line of a flash drum (20, P=1 atm, T=65° C.). The overhead stream from 20 enters the separation column (28), which has about 80 theoretical stages, and a vapor-liquid equilibrium may be achieved at a theoretical stage. The overhead condenser is cooled with cooling water and the reboiler is heated with LP steam. The reflux ratio is set to 30. The overhead condenser is operated at 1 atm and the process stream temperature is around 50° C. The bottom temperature is about 64° C. The distillate is mixed with the feed stream to the front end of the reactor. The bottom stream of 28 is collected as the product stream. The product is analyzed to be 99.9% 1233yd(Z) and 0.1% 1233yd(E).

### Example 5—Isomerization of 1233yd(Z) to 1233yd(E)

The process flow diagram is shown in FIG. 3. A liquid stream of 92% 1233yd(Z) and 8% 1233yd(E) (ambient condition) is pumped (flow rate: 1.3 kg/hr) through a series of heat exchangers to T=380° C. The heated vapor stream is fed into an electric heater reactor (84, 1″(ID)×72″(L)) which has an immersion heating element 86. The temperature of the reactor is measured using a thermal couple on the end of the heating element. The stream enters from the top of the reactor and flows out from the bottom of the reactor.

The effluent from reactor 84 flows through a dry ice trap 90 and a caustic scrubber 92 before it is collected as the reactor product stream 94. The product fractions of the reactor product stream and reactor temperature are listed in Table 1. As shown, The reactor product stream after a single pass through the electric heater reactor includes less than 75%, or less than 72%, or less than 70%, or less than 68% 1233yd(Z), resulting in a conversion of HCFO-1233yd(Z) to HCFO-1233yd(E) of greater than about 17%, or greater than about 20%, or greater than about 22%, or greater than about 24%, or within any range encompassing any two of these values as endpoints.

TABLE 15
Example 5 Temperature and Products

| Electric heater | Reactor product |
|---|---|
| reactor | compositions (mol %) |
| temperature (° C.) | (single pass) |
| 350 | 1233yd(Z) (72.6%), 1233yd(E) 27.3% |
| 400 | 1233yd(Z) (70.8%), 1233yd(E) 29.2% |
| 450 | 1233yd(Z) (69.3%), 1233yd(E) 30.7% |
| 500 | 1233yd(Z) (67.8%), 1233yd(E) 32.2% |

The reactor product may be further separated by batch distillation to separate 1233yd(E) and 1233yd(Z). The stream containing 1233yd(Z) is fed into the electric heater reactor to boost the overall conversion. The process may be repeated multiple times to get the overall conversion of 1233yd(Z) to HCFO-1233yd(E) to be greater than about 90%, greater than about 95%, greater than about 97%, or within any range encompassed by two of the foregoing values as endpoints.

### Example 6—Isomerization of 1233yd(Z) to 1233yd(E)

The process flow diagram is shown in FIG. 4. A liquid stream of 92% 1233yd(Z) and 8% 1233yd(E) (ambient condition) is pumped (flow rate: 1.3 kg/hr) through a series of heat exchangers. The heated vapor stream is fed into a tubular reactor (104, 1″(ID)×60″(L)). The reactor is pre-heated and operated under an isothermal condition (T=380° C.). The effluent from 104 passes through heat exchangers to recover heat. Light impurities are removed from the vapor line of a knockout container (110, P=1 atm, T=20° C.). Heavy impurities are removed from the bottom liquid line of a flash drum (116, P=1 atm, T=65° C.). The overhead stream from 116 enters the separation column (124), which has about 80 theoretical stages, and a vapor-liquid equilibrium may be achieved at a theoretical stage. The overhead condenser is cooled with cooling water and the reboiler is heated with LP steam. The reflux ratio is set to 30. The overhead condenser is operated at 1 atm and the process stream temperature is around 50° C. The bottom temperature is about 63.8° C. The bottom stream is mixed with the feed stream to the front end of the reactor. The distillate stream from 124 is collected as the product stream 138. The product is analyzed to include 99.8% 1233yd(E) and 0.2% 1233yd(Z).

TABLE 16
Summary of Examples 1-6

| Example# | Feed | Reactions | Configuration | Reactor | Catalyst | T (reactor) |
|---|---|---|---|---|---|---|
| 1 | 99.9% 1233yd(E) | 1233yd(E) ↔ | Reactor + | 1″ × 158″ | Yes | 220° C. |
| | 0.1% 1233yd(Z) | 1233yd(Z) | column + | | | |
| | | | recycle | | | |
| 2 | 92% 1233yd(E), | 1233yd(E) ↔ | Reactor + | 1″ × 158″ | Yes | 220° C. |
| | 8% 1233yd(Z) | 1233yd(Z) | column + | | | |
| | | | recycle | | | |
| 3 | 9% 1233yd(E), | 1233yd(E) ↔ | Column + | 1″ × 120″ | Yes | 220° C. |
| | 91% 1233yd(Z) | 1233yd(Z) | reactor + | | | |
| | | | recycle | | | |
| 4 | 99.9% 1233yd(E) | 1233yd(E) ↔ | Reactor + | 1″ × 315″ | No | 250° C. |
| | 0.1% 1233yd(Z) | 1233yd(Z) | column + | | | |
| | | | recycle | | | |
| 5 | 92% 1233yd(Z), | 1233yd(Z) ↔ | Reactor only | 1″ × 72″ | No | 350° C.- |
| | 8% 1233yd(E) | 1233yd(E) | | | | 500° C. |
| 6 | 92% 1233yd(Z), | 1233yd(Z) ↔ | Reactor + | 1″ × 72″ | No | 420° C. |
| | 8% 1233yd(E) | 1233yd(E) | column + | | | |
| | | | recycle | | | |

### Example 7—Isomerization of 1233yd(E) to 1233yd(Z)

The process of Examples 1-3 above are repeated with catalysts listed below in Table 17. Similar conversion and yield of the product 1233yd(Z) are achieved.

TABLE 17
Catalysts used for Example 7

| Catalyst | Cocatalyst |
|---|---|
| chromium oxide (Cr₂O₃) | None |
| chromium oxyfluorides (CrOₓF_{y}) | None |
| chromium halide (CrX₂ or CrX₃) | None |
| Cr₂O₃ | Ni |
| Cr₂O₃ | Zn |
| Cr₂O₃ | Co |
| Cr₂O₃ | Mn |
| Cr₂O₃ | Mg |
| alumina | None |
| iron oxide | None |
| magnesium oxide | None |
| zinc oxide | None |
| nickel oxide | None |
| cobalt oxide | None |
| aluminum fluoride | None |
| iron fluoride | None |
| magnesium fluoride | None |
| zinc fluoride | None |
| nickel fluoride | None |
| cobalt fluoride | None |
| fluorinated alumina | None |
| fluorinated iron oxide | None |
| fluorinated magnesium oxide | None |
| fluorinated nickel oxide | None |
| fluorinated cobalt oxide | None |
| aluminum chloride (AlCl₃) | None |

### Aspects

Aspect 1 is a method for producing HCFO-1233yd(Z), comprising: providing a reactant stream containing at least 50% HCFO-1233yd(E), vaporizing at least a portion of HCFO-1233yd(E) in the reactant stream, converting at least a portion of HCFO-1233yd(E) to HCFO-1233yd(Z) in a reactor, and separating and recovering a composition comprising HCFO-1233yd(Z) or a mixture of HCFO-1233yd(E) and HCFO-1233yd(Z).

Aspect 2 is the method of Aspect 1, wherein the conversion step is conducted in the presence of a catalyst.

Aspect 3 is the method of Aspect 2, wherein the catalyst is selected from the group consisting of a chromium-based catalyst, a promoted chromium-based catalyst, and a non-chromium-based catalyst.

Aspect 4 is the method of Aspect 3, wherein the chromium-based catalyst is chromium oxide; wherein the chromium oxide is fluorinated.

Aspect 5 is the method of Aspect 3, wherein the chromium-based catalyst is chromium oxyfluoride (CrOₓF_{y}), wherein x is greater than 0 and less than 1.5; wherein y is greater than zero and less than 3.

Aspect 6 is the method of Aspect 3, wherein the promoted chromium-based catalyst comprises a co-catalyst selected from the group consisting of Ni, Zn, Co, Mn, Mg, and mixtures hereof.

Aspect 7 is the method of Aspect 6, wherein the co-catalyst content is between 0.1% and 20% based on the total weight of the promoted chromium-based catalyst.

Aspect 8 is the method of any one of Aspects 3, 6, or 7, wherein the promoted chromium-based catalyst comprises zinc oxide and chromium oxide (ZnO—Cr₂O₃).

Aspect 9 is the method of Aspect 3, wherein the non-chromium-based catalyst is selected from the group consisting of: alumina, iron oxide, magnesium oxide, zinc oxide, nickel oxide, cobalt oxide, aluminum fluoride; iron fluoride, magnesium fluoride, zinc fluoride, nickel fluoride, cobalt fluoride, fluorinated alumina; fluorinated iron oxide, fluorinated magnesium oxide, fluorinated nickel oxide, fluorinated cobalt oxide, and mixtures thereof.

Aspect 10 is the method of any one of Aspects 2-9, wherein the reactor is a tubular reactor.

Aspect 11 is the method of any one of Aspects 1-10, wherein the conversion step is carried out at a temperature of from about 50° C. to about 600° C.

Aspect 12 is the method of any one of Aspects 1-11, wherein the conversion step is carried out at a temperature of from about 100° C. to about 500° C.

Aspect 13 is the method of any one of Aspects 1-12, wherein the conversion step is carried out at a temperature of from about 150° C. to about 400° C.

Aspect 14 is the method of any one of Aspects 1-13, wherein the conversion step is carried out at a temperature of from about 200° C. to about 350° C.

Aspect 15 is the method of any one of Aspects 1-14, wherein the conversion step is carried out at a pressure of from about 10 psig to about 100 psig.

Aspect 16 is the method of any one of Aspects 1-15, wherein the conversion step achieves a selectivity to HCFO-1233yd(Z) of at least 30%.

Aspect 17 is the method of any one of Aspects 1-16, wherein the conversion step achieves a conversion of HCFO-1233yd(E) to HCFO-1233yd(Z) of at least 30%.

Aspect 18 is a composition produced from the method of any one of Aspects 1-17, comprising: cis-CHF₂CF═CHCl (HCFO-1233yd(Z)) present in an amount of at least 95 wt. %; and trans-CHF₂CF═CHCl (HCFO-1233yd(E)) present in an amount of less than 5 wt. %, based on a total weight of the composition.

Aspect 19 is the composition of Aspect 18, comprising: cis-CHF₂CF═CHCl (HCFO-1233yd(Z)) present in an amount of at least 97 wt. %; and trans-CHF₂CF═CHCl (HCFO-1233yd(E)) present in an amount of less than 3 wt. %, based on a total weight of the composition.

Aspect 20 is the composition of Aspect 19, comprising: cis-CHF₂CF═CHCl (HCFO-1233yd(Z)) present in an amount of at least 99 wt. %; and trans-CHF₂CF═CHCl (HCFO-1233yd(E)) present in an amount of less than 1 wt. %, based on a total weight of the composition.

Aspect 21 is a method for producing trans-HCFO-1233yd(E), comprising: providing a reactant stream containing at least 50% HCFO-1233yd(Z), vaporizing at least a portion of HCFO-1233yd(Z) in the reactant stream, converting at least a portion of HCFO-1233yd(Z) to HCFO-1233yd(E) in a reactor, and separating and recovering a composition comprising HCFO-1233yd(E) or a mixture of HCFO-1233yd(E) and HCFO-1233yd(Z).

Aspect 22 is the method of Aspect 21, wherein the reactor is a tubular reactor.

Aspect 23 is the method of Aspect 21, wherein the reactor is an electric heater reactor.

Aspect 24 is the method of Aspect 23, wherein the electric heater reactor comprises a metal alloy wire and a compacted metal oxide material.

Aspect 25 is the method of any one of Aspects 21-24, wherein the conversion step is carried out at a temperature of from about 200° C. to about 800° C.

Aspect 26 is the method of any one of Aspects 21-25, wherein the conversion step is carried out at a temperature of from about 250° C. to about 700° C.

Aspect 27 is the method of any one of Aspects 21-26, wherein the conversion step is carried out at a temperature of from about 300° C. to about 600° C.

Aspect 28 is the method of any one of Aspects 21-27, wherein the conversion step is carried out at a temperature of from about 350° C. to about 420° C.

Aspect 29 is a composition produced from the method of any one of Aspects 21-28, comprising: trans-CHF₂CF═CHCl (HCFO-1233yd(E)) present in an amount of at least 95 wt. %; and cis-CHF₂CF═CHCl (HCFO-1233yd(Z)) present in an amount of less than 5 wt. %, based on a total weight of the composition.

Aspect 30 is a composition produced from the method of any one of Aspects 21-28, comprising: trans-CHF₂CF═CHCl (HCFO-1233yd(E)) present in an amount of at least 97 wt. %; and cis-CHF₂CF═CHCl (HCFO-1233yd(Z)) present in an amount of less than 3 wt. %, based on a total weight of the composition.

Aspect 31 is a method for producing cis-HCFO-1233yd(Z), comprising: providing a reactant composition containing at least 50% HCFO-1233yd(E); vaporizing at least a portion of HCFO-1233yd(E) in the reactant composition; converting at least a portion of HCFO-1233yd(E) to HCFO-1233yd(Z) in a reactor at a temperature of from about 50° C. to about 600° C.; and separating and recovering a product composition comprising: HCFO-1233yd(Z)) in an amount of at least 95 wt. %; and HCFO-1233yd(E)) in an amount of less than 5 wt. %, based on a total weight of the product composition.

Aspect 32 is the method of Aspect 31, wherein the converting step is conducted in the presence of a catalyst.

Aspect 33 is the method of 32, wherein the catalyst is selected from the group consisting of fluorinated aluminum chloride, a chromium-based catalyst, and a promoted chromium-based catalyst.

Aspect 34 is the method of Aspect 32 or 33, wherein the catalyst is fluorinated aluminum chloride.

Aspect 35 is the method of Aspect 32 or 33, wherein the catalyst is fluorinated chromium oxide.

Aspect 36 is the method of Aspect 32 or 33, wherein the catalyst comprises zinc oxide and chromium oxide (ZnO—Cr₂O₃).

Aspect 37 is the method of any one of Aspects 31 to 36, wherein the converting step is carried out at a temperature of from about 100° C. to about 500° C.

Aspect 38 is the method of any one of Aspects 31 to 37, wherein the converting step is carried out at a temperature of from about 150° C. to about 400° C.

Aspect 39 is the method of any one of Aspects 31 to 38, wherein the converting step is carried out at a temperature of from about 200° C. to about 350° C.

Aspect 40 is the method of any one of Aspects 31 to 39, wherein the converting step is carried out at a pressure of from about 10 psig to about 100 psig.

Aspect 41 is the method of any one of Aspects 31 to 40, wherein the converting step achieves a selectivity to HCFO-1233yd(Z) of at least 30%.

Aspect 42 is the method of any one of Aspects 31 to 41, wherein the converting step achieves a conversion of HCFO-1233yd(E) to HCFO-1233yd(Z) of at least 30%.

Aspect 43 is the method of any one of Aspects 31 to 42, wherein the product composition comprises: HCFO-1233yd(Z) present in an amount of at least 97 wt. %; and HCFO-1233yd(E) present in an amount of less than 3 wt. %, based on a total weight of the product composition.

Aspect 44 is the method of any one of Aspects 31 to 43, wherein the product composition comprises: HCFO-1233yd(Z) present in an amount of at least 99 wt. %; and HCFO-1233yd(E) present in an amount of less than 1 wt. %, based on a total weight of the product composition.

Aspect 45 is a method for producing trans-HCFO-1233yd(E), comprising: providing a reactant composition containing at least 50% HCFO-1233yd(Z); vaporizing at least a portion of HCFO-1233yd(Z) in the reactant composition; converting at least a portion of HCFO-1233yd(Z) to HCFO-1233yd(E) in a reactor at a temperature of from about 200° C. to about 800° C.; and separating and recovering a product composition comprising: HCFO-1233yd(E) in an amount of at least 95 wt. %; and HCFO-1233yd(Z) in an amount of less than 5 wt. %, based on a total weight of the product composition.

Aspect 46 is the method of Aspect 45, wherein the reactor is an electric heater reactor.

Aspect 47 is the method of Aspect 45 or 46, wherein the converting step is carried out at a temperature of from about 250° C. to about 700° C.

Aspect 48 is the method of any one of Aspects 45 to 47, wherein the converting step is carried out at a temperature of from about 300° C. to about 600° C.

Aspect 49 is the method of any one of Aspects 45 to 48, wherein the converting step is carried out at a temperature of from about 350° C. to about 420° C.

Aspect 50 is the method of any one of Aspects 45 to 49, wherein the product composition comprises: HCFO-1233yd(E) in an amount of at least 97 wt. %; and HCFO-1233yd(Z) in an amount of less than 3 wt. %, based on a total weight of the product composition.

What is claimed is:

1. A method for producing cis-HCFO-1233yd(Z), comprising:
providing a reactant composition containing at least 50% HCFO-1233yd(E);
vaporizing at least a portion of HCFO-1233yd(E) in the reactant composition;
converting at least a portion of HCFO-1233yd(E) to HCFO-1233yd(Z) in a reactor at a temperature of from about 50° C. to about 600° C.; and
separating and recovering a product composition comprising:
HCFO-1233yd(Z)) in an amount of at least 95 wt. %; and
HCFO-1233yd(E)) in an amount of less than 5 wt. %, based on a total weight of the product composition.

2. The method of claim 1, wherein the converting step is conducted in the presence of a catalyst.

3. The method of claim 2, wherein the catalyst is selected from the group consisting of fluorinated aluminum chloride, a chromium-based catalyst, and a promoted chromium-based catalyst.

4. The method of claim 3, wherein the catalyst is fluorinated aluminum chloride.

5. The method of claim 3, wherein the catalyst is fluorinated chromium oxide.

6. The method of claim 3, wherein the catalyst comprises zinc oxide and chromium oxide (ZnO—Cr₂O₃).

7. The method of claim 1, wherein the converting step is carried out at a temperature of from about 100° C. to about 500° C.

8. The method of claim 1, wherein the converting step is carried out at a temperature of from about 150° C. to about 400° C.

9. The method of claim 1, wherein the converting step is carried out at a temperature of from about 200° C. to about 350° C.

10. The method of claim 1, wherein the converting step is carried out at a pressure of from about 10 psig to about 100 psig.

11. The method of claim 1, wherein the converting step achieves a selectivity to HCFO-1233yd(Z) of at least 30%.

12. The method of claim 1, wherein the converting step achieves a conversion of HCFO-1233yd(E) to HCFO-1233yd(Z) of at least 30%.

13. The method of claim 1, wherein the product composition comprises:
HCFO-1233yd(Z) present in an amount of at least 97 wt. %; and
HCFO-1233yd(E) present in an amount of less than 3 wt. %, based on a total weight of the product composition.

14. The method of claim 1, wherein the product composition comprises:
HCFO-1233yd(Z) present in an amount of at least 99 wt. %; and
HCFO-1233yd(E) present in an amount of less than 1 wt. %, based on a total weight of the product composition.

15. A method for producing trans-HCFO-1233yd(E), comprising:
providing a reactant composition containing at least 50% HCFO-1233yd(Z);
vaporizing at least a portion of HCFO-1233yd(Z) in the reactant composition;
converting at least a portion of HCFO-1233yd(Z) to HCFO-1233yd(E) in a reactor at a temperature of from about 200° C. to about 800° C.; and
separating and recovering a product composition comprising:
HCFO-1233yd(E) in an amount of at least 95 wt. %; and
HCFO-1233yd(Z) in an amount of less than 5 wt. %, based on a total weight of the product composition.

16. The method of claim 15, wherein the reactor is an electric heater reactor.

17. The method of claim 15, wherein the converting step is carried out at a temperature of from about 250° C. to about 700° C.

18. The method of claim 15, wherein the converting step is carried out at a temperature of from about 300° C. to about 600° C.

19. The method of claim 15, wherein the converting step is carried out at a temperature of from about 350° C. to about 420° C.

20. The method of claim 15, wherein the product composition comprises:
HCFO-1233yd(E) in an amount of at least 97 wt. %; and
HCFO-1233yd(Z) in an amount of less than 3 wt. %, based on a total weight of the product composition.
